(19) United States
(12) Patent Application Publication
(10) Pub. No.: US 2024/0382554 A1
(43) Pub. Date: Nov. 21, 2024

(54) METHODS OF PREPARING HIGH GINGEROL GINGER EXTRACTS

(71) Applicant: SpecNova LLC, Boca Raton, FL (US)

(72) Inventor: Sebastian Balcombe, Evergreen, CO (US)

(21) Appl. No.: 18/667,554

(22) Filed: May 17, 2024

Related U.S. Application Data
(60) Provisional application No. 63/467,171, filed on May 17, 2023.

(51) Int. Cl. A61K 36/9068; A61K 9/16; A61K 31/12; C12P 5/00

(52) U.S. Cl. CPC A61K 36/9068; A61K 9/1652; A61K 9/1694; A61K 31/12; C12P 5/005; A61K 2236/333; A61K 2236/37; A61K 2236/51

(57) ABSTRACT

Disclosed herein is a method for preparing a ginger extract from ginger rhizome. In certain embodiments, the method includes subjecting the ginger rhizome to a supercritical CO₂ extraction to produce a gingerol fraction and an unextracted fraction; then extracting the unextracted fraction through refluxing ethanol extraction to produce an ethanol extracted fraction and unextracted fiber fraction; then fermenting the unextracted fiber fraction with yeast to produce fermented fibers; and finally combining the gingerol fraction the ethanol extracted fraction, fermented fiber fraction and gum Arabic and homogenizing to form a ginger extract homogenate and spry drying the ginger extract homogenate to produce a ginger extract powder. fermentation with yeast, combining the extracts, and homogenizing with gum arabic, and spray drying to form a ginger extract powder.

### CROSS-REFERENCE TO RELATED APPLICATION(S)

This application claims priority to U.S. Provisional Application No. 63/467,171, filed May 17, 2023, and entitled “METHODS OF PREPARING HIGH GINGEROL GINGER EXTRACTS,” which is hereby incorporated by reference in its entirety under 35 U.S.C. § 119 (e).

### BACKGROUND

Ginger (Zingiber officinale Roscoe) is a common and widely used spice. It is rich in various chemical constituents, including phenolic compounds, terpenes, polysaccharides, lipids, organic acids, and raw fibers. The health benefits of ginger are mainly attributed to its phenolic compounds, such as gingerols, paradols and shogaols. An extract of the rhizome of the perennial plant Zingiber officinale with potential antineoplastic activity. Ginger is a well-known condiment used in traditional medicine and as a dietary supplement. Ginger, also known as Zingiber officinale and belonging to the family Zingiberaceae, is a flowering plant whose rhizome is the most utilized part as a spice and medicine. There is a plethora of uses for ginger due to the presence of various chemical constituents. The ginger rhizome has traditionally been utilized for treating illnesses like cholera, cold, diarrhea, nausea and abdominal pain, toothache, hypertension, hemorrhage, rheumatoid arthritis, and 3 several others. It has also been used as an antiemetic, diuretic, and carminative in other cultures too. The dried form of the rhizome is also attributed to prevent nausea and vomiting in motion sickness and for the treatment of spasmodic gastrointestinal complaints involving bloating and flatulence. Numerous preclinical studies have been conducted to verify its anti-cancer, anti-oxidative and anti-inflammatory activities. There is a need in the art for method of preparing ginger extracts enriched in the ginger compounds associated with health benefits.

### BRIEF SUMMARY

Disclosed herein is a method for preparing a ginger extract from ginger rhizome. In certain embodiments, the method includes subjecting the ginger rhizome to a supercritical CO₂ extraction to produce a gingerol fraction and an unextracted fraction; then extracting the unextracted fraction through refluxing ethanol extraction to produce an ethanol extracted fraction and unextracted fiber fraction; then fermenting the unextracted fiber fraction with yeast to produce fermented fibers; and finally combining the gingerol fraction the ethanol extracted fraction, fermented fiber fraction and gum Arabic and homogenizing to form a ginger extract homogenate and spry drying the ginger extract homogenate to produce a ginger extract powder. fermentation with yeast, combining the extracts, and homogenizing with gum arabic, and spray drying to form a ginger extract powder.

According to certain embodiments, the supercritical CO₂ extraction is performed at a pressure of from about 225-325 bar. In further embodiments, the supercritical CO₂ extraction is performed at a pressure of about 285 bar.

In certain embodiments, the supercritical CO₂ extraction is performed at a temperature of from about 30-60° C. In further embodiments, the supercritical CO₂ extraction is performed at a temperature of from about 35-50° C. in still further embodiments, the supercritical CO₂ extraction is performed at a temperature of about from 40° C.

According to certain embodiments, the supercritical CO₂ extraction is performed for a period of from about 2-4 hours. In further embodiments, the supercritical CO₂ extraction is performed for a period of about 3 hours.

In certain embodiments, the unextracted fraction is extracted in about 95% ethanol. According to exemplary implementations, the ethanol extraction step is performed for from about 2 to about 4 hours. In further embodiments, the ethanol extraction step is performed for about 3 hours.

According to certain embodiments, the fermentation step is performed for from about 48 to about 96 hours. According to further embodiments, the fermentation step is performed for from about 60 to about 84 hours. In yet further embodiments, the fermentation step is performed for about 72 hours.

According to certain embodiments, the yeast used in the fermentation step is Saccharomyces cerevisiae.

According to certain embodiments, following the fermentation step, the fermented fibers are extracted in water.

In certain implementations, the gingerol extract, ethanol extract and fermented fibers are combined at a ratio of about 7:1:1.5 by weight in the homogenization step.

In certain embodiments, gingerol extract, ethanol extract and fermented fibers are combined with the gum arabic at a ratio of about 1:1 by weight.

According to certain embodiments, the spray dying step is performed at an inlet temperature of about 130° C. and an outlet temperature of about 170° C.

According to certain embodiments, the spray dying step is performed with an air flow rate of about 50 Nm3/h. According to certain further embodiments, the spray drying step is performed with a feed rate of about 3.25 mL/min and an atomization pressure of about 1.5 kg/cm².

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 shows ginger compounds associated present in the instantly disclosed ginger extract, according to certain embodiments.

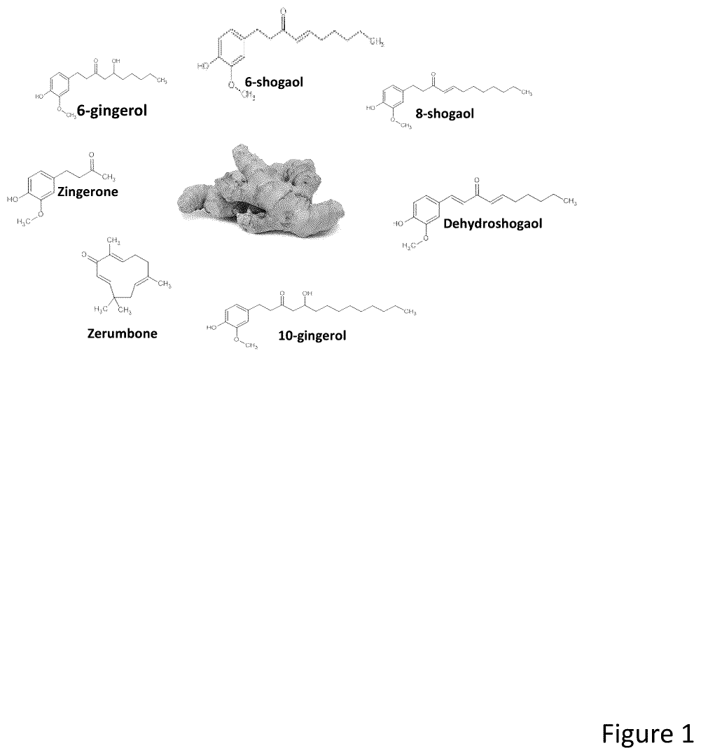

FIG. 2 shows a flow chart of the instantly disclosed extraction process, according to certain embodiments.

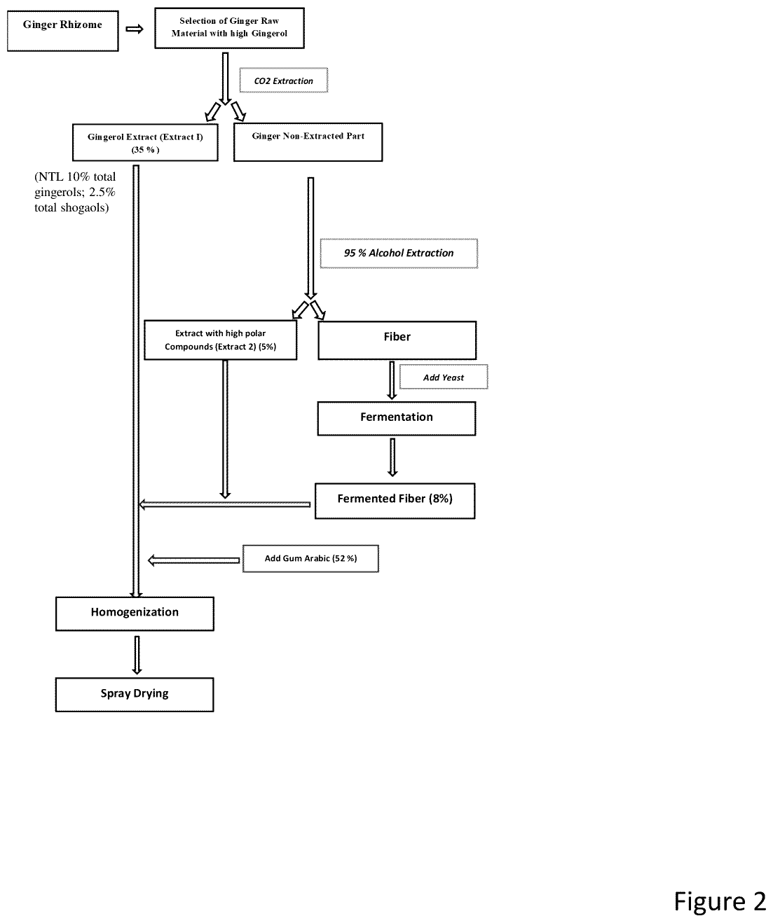

### DETAILED DESCRIPTION

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles” would either completely lack particles, or so nearly completely lack particles that the effect would be the same as if it completely lacked particles. In other words, a composition that is “substantially free of an ingredient or element may still actually contain such item as long as there is no measurable effect thereof.

Admixing or admixed means the formation of a physical combination of two or more elements which may have a uniform or non-uniform composition throughout and includes, but is not limited to, solid mixtures, solutions and suspensions.

Aqueous and aqueous solution mean that water is present but does not require that water be the predominant component. For purposes of illustration and not in limitation, a solution of 90 volume percent of ethylene glycol and 10 volume percent water would be an aqueous solution. Aqueous solutions include liquid media containing dissolved or dispersed components such as, but not in limitation, colloidal suspensions and slurries.

The present disclosure provides a novel process for preparing a ginger extract from ginger rhizome that involves a combination of supercritical CO₂ extraction, ethanol extraction, and fermentation. The process includes subjecting ginger rhizome to supercritical CO₂ extraction to obtain a first extract, subjecting the unextracted ginger to refluxing ethanol extraction to obtain a second extract, subjecting the unextracted fibers to fermentation in the presence of yeast for 72 hours to obtain a third extract, combining the extracts from each of the foregoing extractions, homogenizing the combined extracts with gum arabic, and spray drying the homogenized extract to create a ginger extract powder.

The resulting ginger extract powder obtained by the instantly disclosed process has a high concentration of bioactive compounds, including but not limited to: 6-shogaol, 6-gingerol, and oleoresin, and can be used in a variety of applications, including dietary supplements, functional foods, and pharmaceuticals.

Chemical analysis of ginger shows that it contains over 400 different compounds. The major constituents in ginger rhizomes are carbohydrates (50-70%), lipids (3-8%), terpenes, and phenolic compounds. Terpene components of ginger include zingiberene, β-bisabolene, α-farnesene, β-sesquiphellandrene, and α-curcumene, while phenolic compounds include gingerol, paradols, and shogaol. These gingerols (23-25%) and shogaol (18-25%) are found in higher quantity than others. Besides these, amino acids, raw fiber, ash, protein, phytosterols, vitamins (e.g., nicotinic acid and vitamin A), and minerals are also present.

The aromatic constituents include zingiberene and bisabolene, while the pungent constituents are known as gingerols and shogaols. Other gingerol- or shogaol-related compounds (1-10%), which have been reported in ginger rhizome, include 6-paradol, 1-dehydrogingerdione, 6-gingerdione and 10-gingerdione, 4-gingerdiol, 6-gingerdiol, 8-gingerdiol, and 10-gingerdiol, and diarylheptanoids. The characteristic odor and flavor of ginger are due to a mixture of volatile oils like shogaols and gingerols.

In fresh ginger, gingerols are the major polyphenols, such as 6-gingerol, 8-gingerol, and 10-gingerol. With heat treatment or long-time storage, gingerols can be transformed into corresponding shogaols. After hydrogenation, shogaols can be transformed into paradols. There are also many other phenolic compounds in ginger, such as quercetin, zingerone, gingerenone-A, and 6-dehydrogingerdione.

In certain embodiments, the instantly disclosed process involves four steps: a supercritical CO₂ extraction step; a polar compound extraction step; a fermentation step; and a homogenization and spray drying step.

### Supercritical Carbon Dioxide Extraction

Supercritical CO₂ extraction is a technique that uses carbon dioxide in its supercritical state as a solvent to extract compounds from the plant material. This method is known for its high selectivity and efficiency in extracting volatile and non-polar compounds. According to certain embodiments, ginger rhizomes with a high gingerol are specifically selected for treatment in the instantly disclosed process.

Selected ginger rhizomes are in turn exposed to supercritical CO₂ extraction using a commercial supercritical fluid extractor. In certain embodiments, the extraction chamber of the supercritical fluid extractor is fill from about 40% to about 85% capacity with ginger rhizomes. In further embodiments, the chamber is filled to about 75% capacity with ginger rhizomes.

The extraction conditions are optimized to obtain a first extract with a high concentration of volatile and non-polar compounds. In certain embodiments, the supercritical extraction is performed at a temperature of from about 30-60° C. In further embodiments, the supercritical extraction is performed at a temperature of from about 35-50° C. In still further embodiments, the supercritical extraction is performed at a temperature of about 40° C. In certain embodiments, supercritical extraction is performed at a pressure range of from about 225-325 bar. In further embodiments, supercritical extraction is performed at a pressure of from about 250-300 bar. In yet further embodiments, supercritical extraction is performed at a pressure of about 285 bar. According to certain embodiments, the supercritical CO₂ extraction is carried out for a period of about 1-5 hours. In further embodiments, the supercritical CO₂ extraction is carried out for a period of about 2-4 hours. In yet further embodiments, the supercritical CO₂ extraction is carried out for a period of about 3 hours.

At or near the completion of the supercritical CO₂ extraction, pressure in the extractor is reduced and an extracted gingerol fraction is collected separators. According to certain embodiments, the separators are fitted with cooling jackets. Oil free CO₂ is cooled and recycled for further processing. Non-extracted materials, in the form of a non-extracted ginger powder is removed for processing the ethanol extraction step. The gingol extract, which in certain embodiments comprises about 40% total gingerols, is stored for further processing in the homogenization/spray drying step.

### Ethanol Extraction

According to certain embodiments, following supercritical CO₂ extraction, the unextracted ginger powder is subjected to refluxing ethanol extraction. In certain implementations, the unextracted ginger powder is soaked in 95% ethanol. In certain embodiments, unextracted ginger powder is soaked for from about 1-5 hours. In further embodiments, unextracted ginger powder is soaked for from about 2-4 hours. In still further embodiments, unextracted ginger powder is soaked for about 3 hours. In certain embodiments, following ethanol extraction, pressure is reduced, and ethanol is removed, in certain embodiments at a temperature of about 50° C. According to certain embodiments, the ethanol extract is then filtered and concentrated using a rotary evaporator. The completion of the ethanol extraction step produces a second extract with a high concentration of polar compounds. In certain embodiments, high polar compounds comprise about 5%. This second extract is stored for use in the homogenization and spray drying step. Non-extracted fibers are separated and treated in the fermentation step.

### Fermentation

According to certain embodiments, following refluxing ethanol extraction, the unextracted fibers after the are subjected to fermentation in the presence of yeast. According to certain embodiments, the fermentation step is carried out for a period of about 48-96 hours. In further embodiments the fermentation step is carried out for a period of about 60-84 hours. In yet further embodiments, the fermentation step is carried out for a period of about 72 hours. According to certain embodiments, the fermentation is carried out at a temperature range of 25-30° C. and a pH range of 5.5-6.5. According to certain embodiments, the yeast used in the fermentation process is Saccharomyces cerevisiae. The resulting fermented fibers are used in the homogenization and spray drying step. According to certain embodiments, prior to the homogenization step, the fermented fibers are subjected to an extraction in an aqueous solution. In exemplary implementations, the aqueous solution is water.

### Homogenization and Spray Drying

According to certain embodiments following the fermentation step, extracts from the supercritical CO₂ (gingerol extract) and ethanol extraction steps are combined with the fermented fibers and homogenized. In certain implementations, the gingerol extract, ethanol extract and fermented fibers are combined at a ratio of about 7:1:1.5 by weight. In exemplary implementations, the foregoing extracts are combined with gum arabic. In certain exemplary implementations of these embodiments, the combined ginger extracts and fermented are combined with gum Arabic at a ratio of about 1:1 by weight.

According to certain embodiments, following homogenization, spray drying is performed to create a ginger extract powder. In certain implementations, spray drying is performed with 130° C. inlet temperature, 170° C. outlet temperature, 50 Nm3/h air flow rate, 3.25 ml/min feed rate, and 1.5 kg/cm² atomization pressure.

### EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1

### Step: 1

A 100 kg of ginger rhizome with a high gingerol content were carefully selected and subjected to super critical carbon dioxide extraction. For that, the ginger raw material was filled into the upper compartment of the extraction apparatus up to three-fourths of the volume. The CO₂ was then pumped into the extraction vessel via a pump. The extraction was performed at 40°±2° C., and CO₂ pressure of the extractor was 280 bar. Using pressure reduction, gingerol dispersed in supercritical CO₂ was separated from CO₂ and collected in separators. Separators were fitted with cooling jackets. In the storage tank, oil-free CO₂ was cooled and recycled. After carrying out the extraction for 3 h, CO₂ was released slowly through a Teflon tube to yield 5.5 kg of gingerol extract (with 40% total gingerols) and 88 kg of non-extracted ginger powder.

### Step: 2

In step 2, the 88 kg of the ginger's non extracted portion soaked in 95% ethanol (440 L) and subjected to extraction by refluxing for 3 hours. After extraction, the solvent was removed at reduced pressure at 50 degrees. The concentrated extract yielded 1.1 Kg.

### Step: 3

The separated fibers were fermented in the presence of yeast about 72 hours during the third stage, resulting in the formation of fermented fibers.

### Step: 4

The ginger extract powder was created by homogenizing 52 kg of gum arabic with the gingerol extract (35 Kg, made using multiple extractions) extracts with high polar components (5 Kg), and fermented fibers (8 Kg) in the last phase. Afterwards, spray drying was done by applying 130° C. inlet temperature, 170° C. outlet temperature, 50 Nm3/h air flow rate, 3.25 ml/min feed rate, and 1.5 kg/cm² atomization pressure.

Although the disclosure has been described with reference to preferred embodiments, persons skilled in the art will recognize that changes may be made in form and detail without departing from the spirit and scope of the disclosed apparatus, systems and methods.

What is claimed is:

1. A method for preparing a ginger extract from ginger rhizome comprising:
a) subjecting the ginger rhizome to a supercritical CO₂ extraction to produce a gingerol fraction and an unextracted fraction;
b) extracting the unextracted fraction through refluxing ethanol extraction to produce an ethanol extracted fraction and unextracted fiber fraction;
c) fermenting the unextracted fiber fraction with yeast to produce fermented fibers; and
d) combining the gingerol fraction the ethanol extracted fraction, fermented fiber fraction and gum Arabic and homogenizing to form a ginger extract homogenate and spry drying the ginger extract homogenate to produce a ginger extract powder.

2. The method of claim 1, wherein the supercritical CO₂ extraction is performed at a pressure of from about 225-325 bar.

3. The method of claim 2, wherein the supercritical CO₂ extraction is performed at a pressure of about 285 bar.

4. The method of claim 1, wherein the supercritical CO₂ extraction is performed at a temperature of from about 30-60° C.

5. The method of claim 4, wherein the supercritical CO₂ extraction is performed at a temperature of from about 35-50° C.

6. The method of claim 5, wherein the supercritical CO₂ extraction is performed at a temperature of about 40° C.

7. The method of claim 1, wherein the supercritical CO₂ extraction is performed for a period of from about 2-4 hours.

8. The method of claim 7, wherein the supercritical CO₂ extraction is performed for a period of about 3 hours.

9. The method of claim 1, wherein the unextracted fraction is extracted in about 95% ethanol.

10. The method of claim 1, wherein the ethanol extraction step is performed for from about 2-4 hours.

11. The method of claim 10, wherein the ethanol extraction step is performed for about 3 hours.

12. The method of claim 1, wherein the fermentation step is performed for from about 48-96 hours.

13. The method of claim 12, wherein the fermentation step is performed for from about 60-84 hours.

14. The method of claim 13, wherein the fermentation step is performed for about 72 hours.

15. The method of claim 1, wherein the yeast used in the fermentation step is Saccharomyces cerevisiae.

16. The method of claim 12, wherein following the fermentation step, the fermented fibers are extracted in water.

17. The method of claim 1, wherein the gingerol extract, ethanol extract and fermented fibers are combined at a ratio of about 7:1:1.5 by weight in the homogenization step.

18. The method of claim 1, wherein the gingerol extract, ethanol extract and fermented fibers are combined with the gum arabic at a ratio of about 1:1 by weight.

19. The method of claim 1, wherein the spray dying step is performed at an inlet temperature of about 130° C. and an outlet temperature of about 170° C.

20. The method of claim 1, wherein the spray dying step is performed with an air flow rate of about 50 Nm3/h and wherein the spray drying step is performed with a feed rate of about 3.25 mL/min and an atomization pressure of about 1.5 kg/cm².
